(19) United States
(12) Patent Application Publication
(10) Pub. No.: US 2021/0186718 A1
(43) Pub. Date: Jun. 24, 2021

(54) STIMULATION DEVICE AND PROSTHESIS DEVICE COMPRISING AT LEAST ONE STIMULATION DEVICE FOR STIMULATING NERVE CELL ENDS AND USE OF A VIBRATION GENERATOR FOR STIMULATING NERVE CELL ENDS IN A VIBRATION-DECOUPLED MANNER

(71) Applicant: Saphenus Medical Technology GmbH, Krems an der Donau (AT)

(72) Inventors: Martin Brandstätter, Wien (AT); Bernhard Zach, Wien (AT); Aaron Pitschl, Wien (AT)

(73) Assignee: Saphenus Medical Technology GmbH, Krems an der Donau (AT)

(21) Appl. No.: 17/057,257

(22) PCT Filed: May 21, 2019
(86) PCT No.: PCT/IB2019/054191

§ 371 Date: Nov. 20, 2020

(30) Foreign Application Priority Data
May 30, 2018 (EP) 18175172.8

(51) Int. Cl. A61F 2/68; A61F 2/80

(52) U.S. Cl. CPC A61F 2/68; A61F 2002/6827; A61F 2/80

(57) ABSTRACT

A stimulation device for stimulating reinnervated nerve cell ends, includes: at least one vibration generator, where the at least one vibration generator is connected to at least one decoupling element in a vibration-proof manner. A prosthesis device having at least one stimulation device for stimulating nerve cell ends, in particular nerve area sections typical of the physiognomy, and having a prosthesis shaft as well as at least one vibration generator, where the at least one vibration generator is connected in a positionally fixed manner to the prosthesis shaft via at least one decoupling element.

The invention relates to a stimulation device for stimulating nerve cell ends, a prosthesis device having at least one stimulation device for stimulating nerve cell ends, and a vibration generator stimulating nerve cell ends in a vibration-decoupled manner according to the preambles of the independent claims.

On 8 Jun. 2015, an article appeared in the section Science>Humanity of the online daily newspaper derStandard.at which presented information about a development by Professor Hubert Egger, one of the world's leading prosthetics researchers. The article contained s report on the first leg prosthesis which “feels sympathetically”. It explained in detail how severed nerves in a patient whose leg had been amputated were reactivated and relocated to an area of skin on the leg stump. Since this area of skin is subsequently the location of the reinnervated nerve cell ends, it is particularly sensitive. In this process, the nerve ends were repositioned in such manner that the former foot with its nerve area sections typical of the physiognomy was replicated in this area of skin by the nerve ends that were relocated there. In other words, a nerve area section typical of the physiognomy is a natural nerve area section which is relocated to the surface of a skin area that is actually not typical of it. Consequently, in this skin area the patient felt not (only) the skin (surface) which was there, as usual, but the reinnervated nerve cell ends, that is to say the sole of the foot as it were, which no longer even existed since the leg was amputated.

The stimulators that were used in the solution presented were designed so as to emit a vibration signal directly in the shaft of the prosthesis, to indicate to the patient with a vibration signal through the shaft wall that precisely this section of the artificial sole was now subjected to pressure. The vibrators which were used as stimulators in this arrangement then generated a brief vibration in the shaft of the prosthesis as soon as the pressure sensor coupled to the respective vibrator detected a pressure signal on the corresponding sole area of the artificial sole.

The disadvantage of this arrangement is that the vibration of the vibrators is transferred to the prosthesis shaft, and is consequently propagated in part over the shaft surface as well. This has the effect of diminishing the intended pinpoint precision of the indication, and furthermore the shaft material functions as a resonance body, and consequently a noise generation is audible, which may be perceived as irritating by a user and those around him.

CN 106691814 A discloses a vibrating massage wand with a rotatable rod as vibration generator, which is driven by a motor and causes the housing of the vibrating massage wand to start vibrating. The motor is disposed inside a handle unit of the vibrating massage wand. A coupling element is arranged between the motor and the rotating rod in order to transmit the driving motion to the rotatable rod. A separate damping unit is also arranged on the handle unit to suppress the transmission of vibrations from the housing to the handle unit.

The disadvantage of this solution is that an additional damping unit is needed to reduce the transmission of vibrations to the handle unit of the vibrating massage wand. Species-related stimulation devices are also disclosed in KR 2012 092226 A1 and EP 3 125 574 A1.

WO 2009/014644 A1 discloses an orthopaedic assembly and prosthesis device for a human extremity with a feedback system for alerting a user, wherein the user is alerted, in the event that the human joint is misaligned, for example, with the aid of a vibration generator. In this way, the user is habituated with each misalignment of the joint by means of the vibration from the vibration generator, wherein vibrations are transmitted either in the region of the joint or to another part of the body.

The disadvantage of this known solution is that the vibration generators are arranged immediately inside the orthopaedic assembly and prosthesis device, and the vibrations from the vibration generator act on or directly inside the orthopaedic assembly and prosthesis device. In this case too, this in turn leads to resonance effects and generates noise.

It is the object of the present invention to remedy one or more disadvantages of the related art. In particular, it is intended to create a stimulation device for stimulating nerve cell ends which enables noise to be suppressed while the nerve cells are being stimulated, thereby improving wearing comfort for the person wearing the prosthesis. Further, it is intended to provide a prosthesis device with at least one stimulation device for stimulating nerve cell ends and a vibration generator for stimulating nerve cell ends which remedies one or more of the disadvantages of the related art and is better designed for achieving the intended purpose, namely transmitting signals to innervated nerve ends.

The object is solved with the assemblies defined in the independent claims. Advantageous further developments are presented in the figures, the description and particularly in the dependent patent claims.

The inventive stimulation device for stimulating nerve cell ends is equipped with at least one vibration generator, wherein the at least one vibration generator is connected to at least one decoupling element in a vibration-proof manner. The decoupling element decouples the vibrations or oscillations generated by the vibration generator from its surroundings, from a base, for example, in particular from a prosthesis shaft, and absorbs the vibrations or oscillations, thereby enabling the prevention of noise-carrying transmissions of vibrations or transmissions of oscillation to the surrounding region, to the prosthesis shaft for example. Since no vibrations or oscillations are transmitted, a vibration-proof state exists. In other words, this means that a conventional vibration generator, such as a Vibracall element, which previously typically provided for noise generation and/or made allowance for the generation of a humming noise as a side effect during the vibration process or during the oscillation process as an integral part of its intended use can be used as a noiseless stimulator for nerve cell ends. The use of Vibracall elements is known to cause a mobile phone to vibrate as an entire unit so that a person carrying the phone can be made aware of an incoming call even when he does not hear a ringtone. To ensure that an incoming call in Vibracall mode is still detected even if the mobile phone is not carried in direct contact with the body, the humming sound generated by the resonance vibration of the mobile phone housing is desirable.

The novel stimulation device is designed particularly for stimulating reinnervated nerve cell ends. Reinnervated nerve cell ends are nerve cell ends which have been moved by a physician to a different skin area surface and which—depending on the individual patient and the success of the operation—have high tactile sensitivity auf. Accordingly, they are particularly sensitive to stimulation with the stimulation device. Like all nerves, however, they can also be trained. The invention is also intended to be used for training purposes of such kind.

Preferably a base is present, on which the at least one vibration generator is arranged, and by which the at least one vibration generator is kept stable. In other words, the base makes it possible to mount the at least one vibration generator securely, so that a reproducible stimulation of the nerve cell endings can take place.

The at least one vibration generator is preferably connected to the base in positionally fixed manner via the at least one decoupling element. In this way, the vibrations or oscillations are decoupled from the base. Since the vibrations or oscillations are not transmitted to the base, resonance between the at least one vibration generator and the base cannot be created, with the result that the at least one vibration generator can vibrate or oscillate on the base noiselessly.

The at least one vibration generator is advantageously arranged so as to be adjustable on the base. Then, the spatial position of the at least one vibration generator relative to the base may be changed. The at least one vibration generator may be arranged so as to be displaceable on the base along a first longitudinal axis. Alternatively or additionally thereto, the at least one vibration generator may be arranged on the base so as to be rotatable about a fastening means. Consequently, the at least one vibration generator may be adjusted specifically to individual nerve cell ends, so that the stimulation of the nerve cell ends can be improved further.

At least a section of the at least one vibration generator advantageously has a vibration insulation made of an elastic material such as a rubber material. This vibration insulation prevents the at least one vibration generator from interacting with the base so as to transfer sound. This enables additional decoupling of sound from the base.

Preferably, at least one further decoupling element is present, thereby improving the decoupling of the vibrations or oscillations.

The at least one further decoupling element is preferably connected to the at least one vibration generator. The further decoupling element is typically also arranged between the at least one vibration generator and the base, so that the at least one vibration generator is mounted firmly on the base and/or the at least one vibration generator can be secured symmetrically.

The at least one decoupling element is preferably a spring element. This is a simple way whereby the vibration or oscillation of the at least one vibration generator can be damped relative to the base, so that the noise generated by the vibration or oscillation is suppressed without eliminating the vibration energy at the same time. Typically, a wire such as a steel wire is used as the spring element, wherein the wire must be sufficiently strong and rigid.

In particular, the spring element is constructed in the form of a rod or a spiral, so that the spring element can be fastened easily to the base and/or improved decoupling during vibration or oscillation is enabled.

The at least one decoupling element and the at least one further decoupling element are preferably each spring elements. In such a case, noise generation during vibration or oscillation can be prevented, and improved decoupling may be achieved.

More preferably, at least the at least one decoupling element is electrically conductive, so that the at least one decoupling element is usable as an electrical supply connector for the at least one vibration generator.

In particular, the at least one decoupling element and the at least one further decoupling element are electrically conductive, so that they are usable as electrical supply connectors for the at least one vibration generator. This means that no additional electrical supply connectors are necessary on the at least one vibration generator, which in turn significantly prolongs the operating life of the at least one vibration generator. The spring elements are typically piano wires because they are easy to connect with the at least one vibration generator, they can decouple vibration or oscillation effectively and they are electrically conductive.

Preferably, a supply device is present, with which the at least one vibration generator can be supplied simply with electrical energy. The supply device typically has a rechargeable battery unit or accumulator unit and a charge connector so that the rechargeable battery unit or accumulator unit can be connected to an external energy storage system, for example a supply voltage network, and recharged.

Alternatively or additionally thereto, a control device is present by which the at least one vibration generator may be actuated. The control device typically contains a processor a number of control programs, by which the at least one vibration generator may be actuated systematically.

In particular, the control programs contain control modes with various control commands which are called depending on the respective control mode. In this way, different motion processes, such as a running motion, walking motion, mountain climbing motion may be activated in the control device.

The control device preferably comprises a memory unit, in which the control programs may be stored. In particular, the memory unit is disposed inside the control device in such manner that it can be swapped out and/or the memory capacity can be expanded.

The at least one vibration generator is preferably electrically connected to the supply device and/or the control device, whereby the aforementioned advantages can be realised particularly easily.

More preferably, the at least one vibration generator is equipped with an eccentric element or a vibration element, with which the nerve cell ends are able to be stimulated easily. The eccentric element or the vibration element switch the at least one vibration generator into a vibrating or oscillating state.

The vibration in the vibration generators is generated with an eccentric element due to the changing centripetal force of the eccentric element. Since the centre of mass of the eccentric element is not positioned centrally on the axle on the drive shaft of the vibration generator, an unbalance is created and acts on the smooth running of the drive unit and starts the vibration generator vibrating. The more distant the centre of mass of the eccentric element is from the axis of rotation, the greater the mass and the rotating speed are, and consequently the greater the centripetal force becomes, which in turn increases the amplitude of vibration of the vibration generator.

The vibration or oscillation in vibration generators with a vibration element is generated by the vibration element in that the vibration element is accelerated by a short electromagnetic pulse and then returned to its starting position again by means of a spring unit. The mass moment of inertia created thereby gives rise to a simple harmonic oscillation.

The at least one vibration generator is preferably equipped with a drive motor for driving the eccentric element in rotary manner, so that the eccentric element may be caused to rotate.

Alternatively, the at least one vibration generator has a drive actuator for driving the vibration element in oscillating manner, so that the vibration element may be caused to oscillate harmonically.

The at least one vibration generator preferably has a vibration generator housing, so that the vibrations or oscillations may be transmitted to the surrounding environment and/or the nerve cell ends via the vibration generator housing.

The eccentric element or the vibration element is preferably disposed inside the vibration generator housing. This ensures that no self-supporting movable components are present on the at least one vibration generator, and that despite this vibrations or oscillations can still be transmitted to the surrounding environment and/or the nerve cell ends.

In particular, the vibration generator housing is embodied as a button cell, in which the drive motor as well as the eccentric element or the vibration element are disposed. This allows a simple, compact construction which also enables the oscillation device to function without interference.

More preferably, the at least one vibration generator has a stimulation unit for stimulating nerve cell ends, by which the vibrations or oscillations can be converted effectively into stimulations.

The stimulation unit is preferably constructed in the shape of a spike. This enables the nerve cell ends to be stimulated even more precisely, which in turn possible results in improved and/or more profound stimulation of the nerve cell ends.

Alternatively, the stimulation unit is cylindrical or hemispherical. This enables the stimulation unit to be adapted to different nerve cell end structures so that they can be stimulated efficiently.

Preferably, at least a section of the at least one vibration generator is surrounded by an insulation layer. This ensures that the at least one vibration generator is protected from environmental influences such as moisture, thus prolonging the operating life of the at least one vibration generator.

Preferably, the at least one vibration generator is completely surrounded by the insulation layer. This serves as a simple was to prevent a short circuit in or with the vibration generator housing, for example.

A further aspect of the invention relates to a prosthesis device having at least one stimulation device for delivering information to nerve cell ends, with a prosthesis shaft and with at least one vibration generator. The at least one vibration generator is connected to the prosthesis shaft in positionally fixed manner via at least one decoupling element. This prevents a transmission of the vibration or oscillation from the at least one vibration generator to the prosthesis shaft, which thus enables users or the patient using the prosthesis device to feel the vibration or oscillation at the respective nerve cell end which is to be stimulated, and at the same time is not irritated by the generation of noise at the prosthesis shaft. This in turn improves wearing comfort for the person wearing the prosthesis.

The at least one stimulation device is preferably the stimulation device described in this document, which serves particularly effectively to suppress noise while stimulating the nerve cell ends.

The delivery of information to the nerve cell ends particularly comprises a stimulation of nerve cell ends, in particular nerve area sections typical of the physiognomy, whereby it is possible for the nerve cell ends to be trained.

The at least one decoupling element preferably has a fastening section for fastening the decoupling element to the prosthesis shaft. This makes it possible to hold the at least one vibration generator securely on the prosthesis shaft. The fastening section is typically located on one end of the at least one decoupling element, thereby ensuring effective decoupling during vibration or oscillation and at the same time enabling effective stimulation of the nerve cell ends.

The fastening section preferably comprises at least one fastening means, which fastens at least one of the decoupling elements to the base. Typically, either detachable fastening means such as a plug-in fastener or a screw fastener is used, enabling the stimulation device to be replaced easily and making it simple to service the stimulation device and/or the prosthesis device.

In particular, the prosthesis shaft has a dedicated prosthesis opening for each vibration generator. In this way, nerve cell ends at a distance from each other can be stimulated while the vibration generators do not interfere with each other. In addition, the prosthesis shaft remains dimensionally stable despite multiple prosthesis openings and retains sufficient strength.

A seal insert, made from a silicone material for example, is advantageously arranged between the at least one prosthesis opening and the at least one vibration generator. This helps to prevent the user's limb being pushed outwards through the at least one prosthesis opening, which might result in skin irritations.

The seal insert is advantageously designed to be airtight and/or fluid-tight. This makes it possible to shield the at least one vibration generator from the user's bodily fluids. An airtight seal insert also enables the use of a vacuum prosthesis shafts with the at least one vibration generator, wherein the vacuum prosthesis shaft may be arranged on the user's extremity with the aid of a negative pressure system.

Alternatively or additionally thereto, a non-detachable fastening means such as an adhesive connection may also be used as the fastening means.

In particular, the fastening section comprises a number of fastening means for fastening each of the decoupling elements individually. This enables each of the decoupling elements to be fastened to the prosthesis shaft separately.

More preferably, an electrical supply device is present on the prosthesis shaft, the electrical supply device being connected electrically to the at least one vibration generator. The supply device as described here is typically embodied as a rechargeable battery unit or accumulator unit, so that the at least one vibration generator can be supplied with energy while the prosthesis device is being used by the patient.

In particular, the electrical supply device includes a charge connector so that it may be connected to an external energy supply.

In particular, the electrical supply device includes a charge coupler, with which the electrical supply device can be recharged inductively using a separate charging unit. This enables the electrical supply device to be recharged wirelessly. For example, the charge coupler is equipped with at least one magnet for inductive charging of the electrical supply device.

The at least one decoupling element is preferably arranged between the electrical supply device and the at least one vibration generator and connected electrically to the electrical supply device and the at least one vibration generator. This enables the energy from the supply device to be delivered to the at least one vibration generator via the decoupling element, so that no additional supply lines are needed for the vibration generator.

More preferably, a control device is present for controlling the at least one vibration generator on the prosthesis shaft, the at least one vibration generator being connected electrically to the control device. In this way, the at least one vibration generator is able to be supplied with stimulation commands which are typically transmitted from the control device to the at least one vibration generator. In this context, the control device is mounted on the prosthesis shaft in such manner that position of the control device serves as a counterbalance as far as possible, and thus affords greater wearing comfort for the prosthesis wearer.

In particular, the control device includes a processor by which the previously described control modes are executable on the prosthesis device.

The prosthesis shaft preferably has at least one positioning section for positioning the at least one vibration generator, wherein the positioning section has at least one prosthesis opening. With this, the at least one vibration generator may be mounted securely on the prosthesis shaft and at the same time transmit a decoupled stimulation through the prosthesis opening to the user's or the prosthesis wearer's nerve cell ends.

The prosthesis device preferably has multiple stimulation devices by which different nerve cell ends on the skin surface may be stimulated.

In particular, each of the multiple stimulation devices has its own fastening section with a fastening means, so that each of the multiple stimulation devices can be positioned individually on the prosthesis device, for example on the prosthesis shaft.

In particular, each of the multiple stimulation devices is connected to the supply device by its own supply lines so that each may be supplied independently of the others, wherein the supply lines are detachably connected to each of the multiple stimulation devices so that each of the multiple stimulation devices is easily replaceable.

A further aspect of the invention relates to the provision of a vibration generator with at least one eccentric element or at least one vibration element (Vibracall*) for the purpose of stimulating nerve cell ends in vibration-decoupled manner from a base, preferably from a prosthesis shaft, in particular for stimulating reinnervated nerve cell ends and/or for delivering information from reinnervated nerve cell ends. This enables particularly efficient transmission of a vibration process or oscillation process to a user's or a patient's nerve cell ends.

At the same time, the at least one vibration generator preferably has at least one decoupling element by which the generation of noise that is irritating for the user or prosthesis wearer can be suppressed, thus improving wearing comfort.

A further aspect of the invention relates to the use of the stimulation device described here as a signal generator in a communication apparatus. Thus, a noiseless, tactile signal generator is produced in a communication apparatus. The signal generator and the at least one vibration generator are not in vibratory resonance with the communication apparatus, so a “humming noise” is not produced by the communication apparatus itself.

The communication apparatus is preferably a portable communication apparatus, in particular a mobile telephone or a tablet. Accordingly, besides the advantages described previously, the user of the portable communication apparatus is alerted in the event of an incoming call or an incoming message by the pinpoint vibration or pinpoint interaction between the at least one vibration generator of the vibration device on/with the user's body or on/with a support surface for the communication apparatus, for example a table support surface. In this respect in particular, the use of a mobile phone or a Tablet is improved, because the incoming calls or incoming messages can be dealt with more discreetly in the presence of third parties.

The communication apparatus preferably has at least one housing opening, in which at least a section of the at least one vibration generator is arranged, as described earlier in this document. This has the effect of improving the locational precision of the information to the user, in particular locationally precise tactile information to the user.

In particular, the at least one vibration generator has a vibration generator housing which in particular is cylindrical in shape, wherein at least a section of the cylindrical surfaces of the cylinder is arranged in the housing opening (vertical arrangement). In this way, the vibrations are transmitted to the user directly in the direction of the vibrations.

In particular, at least a section of the shell surface of the at least one vibration generator is arranged in the housing opening (horizontal arrangement). This enables the at least one vibration generator to be placed in the housing opening easily.

Alternatively or additionally thereto, other geometric bodies also lend themselves to use as vibration generator housings, such as a cuboid vibration generator. With this, the locationally precise information to the user can be optimised for the respective purpose.

More preferably, the communication apparatus is furnished with an insulating arrangement for insulating the at least one vibration generator from the housing opening. This enables the communication apparatus to be disposed on the housing of the communication apparatus entirely without resonance effects.

In particular, the insulating arrangement consists of a soft and/or elastic material. Typically, a readily workable silicone mass with sufficient insulating properties is used for this.

The communication apparatus preferably has a stimulation device with one vibration generator arranged horizontally and a further stimulation device with a vibration generator arranged vertically. This allows two different vibration modes or oscillation modes to be used in the communication apparatus.

The horizontally arranged vibration generator and the vertically arranged vibration generator are preferably each arranged in a separate housing opening in the communication apparatus. This way, the vibrations from the respective vibration generators can be separated.

The communication apparatus preferably has a selection switching unit for selecting the respective vibration generators. The user can then use the selection switching unit to select either the horizontally arranged vibration generator of a vertically arranged vibration generator as the signal generator.

Further advantages, features and particularities of the invention are revealed in the following description, in which exemplary embodiments of the invention are described with reference to the figures.

The list of reference numbers, like the technical content of the patent claims and the figures constitutes part of the disclosure. The figures are described in sequence and in context with the other figures. The same reference signs indicate identical components, reference signs with different indices denote functionally equivalent or similar components.

The figures show diagrammatically:

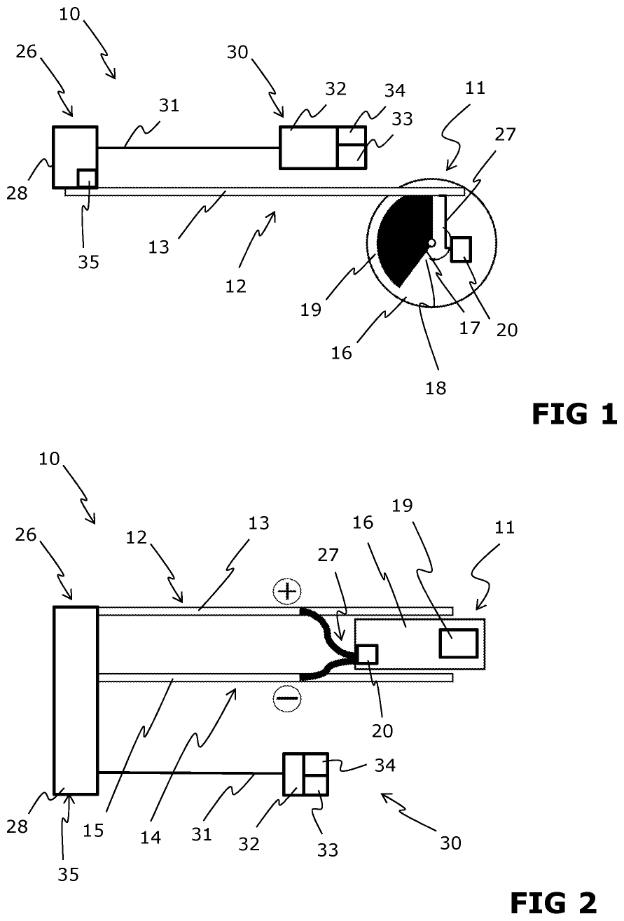

FIG. 1 a side view of a stimulation device according to the invention with a first embodiment of a vibration generator,

FIG. 2 a top view of the stimulation device of FIG. 1,

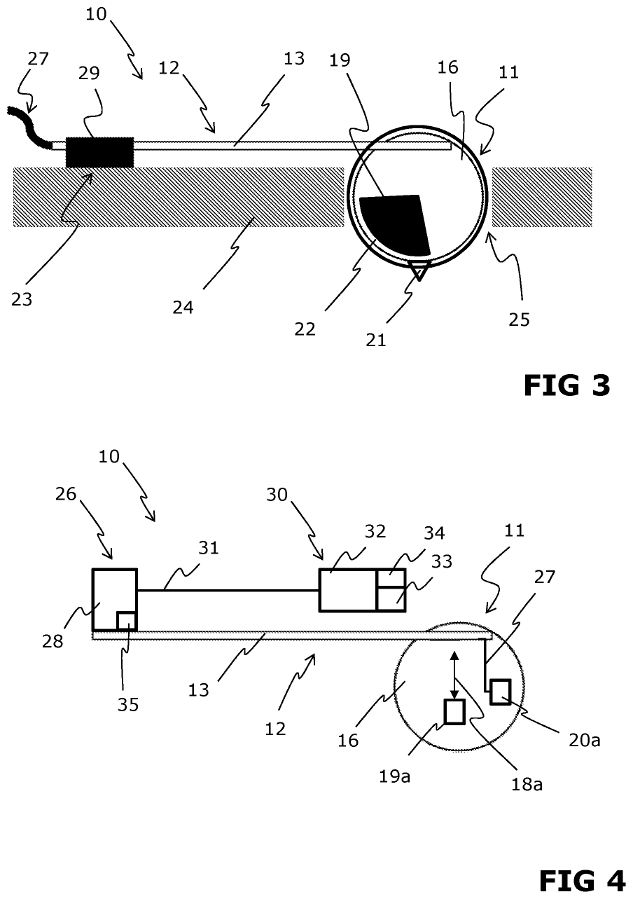

FIG. 3 a side view of the stimulation device of FIG. 1 in a base,

FIG. 4 a side view of the stimulation device of FIG. 1 with a further embodiment of the vibration generator,

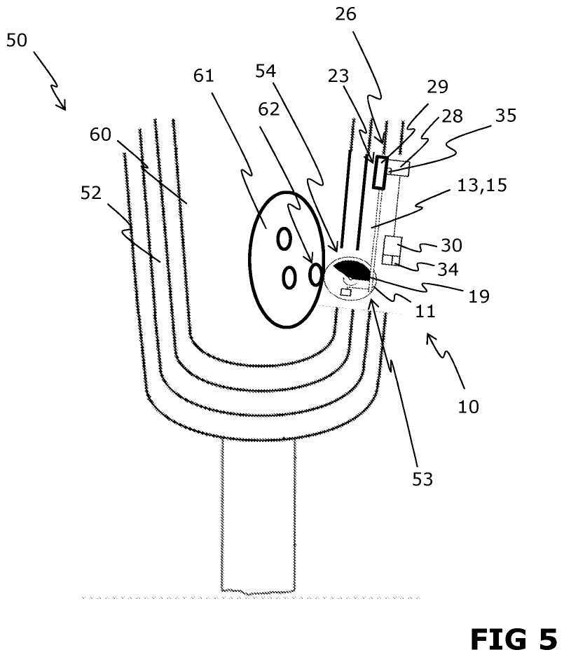

FIG. 5 a perspective view of a prosthesis device according to the invention with a stimulation device according to FIG. 1, and

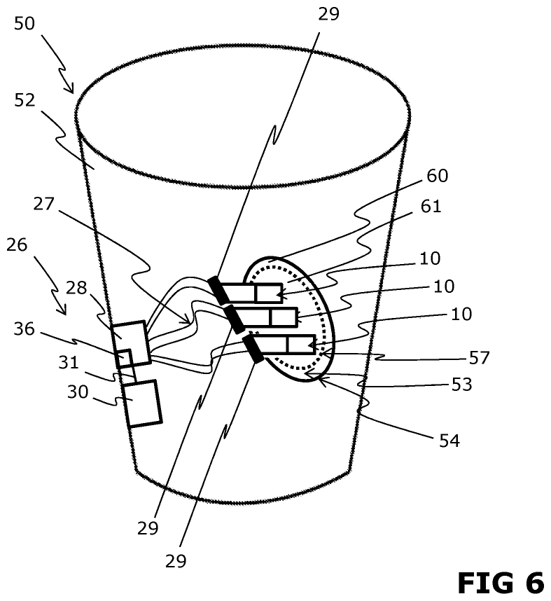

FIG. 6 a perspective view of the prosthesis device of FIG. 5 with multiple stimulation devices according to FIG. 1.

FIG. 1 and FIG. 2 show the stimulation device 10 according to the invention for stimulating nerve cell ends. The stimulation device 10 includes a vibration generator 11 with a vibration generator housing 16, which is connected to a first spring element 13 and a second spring element 15 as decoupling elements 12 and 14. The first spring element 13 and the second spring element 15 are rod-shaped spring wires which are arranged on opposite sides of the vibration generator housing 16. For this purpose, the spring elements 13 and 15 are each attached by one of their ends to the vibration generator housing 16. The spring elements 13 and 15 decouple the vibrations or oscillations of the vibration generator 11 form the surrounding area. An eccentric element 19 is arranged inside the vibration generator housing 16. The eccentric element 19 is mounted such that it is able to turn or rotate in drive direction 18 on the drive shaft 17. The vibration of the vibration generator 11 is created with the eccentric element 19 by the changing centripetal force of the eccentric element 19. Since the centre of mass of the eccentric element 19 is not positioned centrally on the axle on the drive shaft 17 of the vibration generator 11, an unbalance is created which acts on the smooth running of the eccentric element 19 (a running disturbance is created) and starts the vibration generator 11 vibrating. The more distant the centre of mass of the eccentric element 19 is from the axis of rotation of the drive shaft 17, the greater the mass and the rotating speed of the eccentric element 19 are, and consequently the greater the centripetal force becomes, which in turn increases the vibration amplitude of the vibration generator 11. The vibration generator housing 16 of the vibration generator 11 accommodates a drive motor 20 for driving the eccentric elements 19 rotationally. For this purpose, the drive motor 20 is connected to the drive shaft 17. In the configuration shown, the vibration generator 11 is embodied as a button cell. The drive shaft 17, the eccentric element 19 and the drive motor 20 are all arranged inside the vibration generator housing 16, which is in the shape of a cylinder.

The stimulation device 10 is equipped with a supply device 26. The supply device 26 comprises a rechargeable battery unit or an accumulator unit 28 with a charge connector 35 and is connected to one end of each of the first spring element 13 and the second spring element 15. The spring elements 13 and 15 are electrically conductive and supply the drive motor 20 with electrical voltage. At the same time, the electrically conductive first spring element 13 serves as the supply for the positive DC voltage connection and the electrically conductive second spring element 15 serves as the supply line for the negative DC voltage connection of the drive motor 20 (FIG. 2). Supply lines 27 are attached to the spring elements 13 and 15 and connect the drive motor 20 to the battery unit or accumulator unit 28.

The stimulation device 10 includes a control device 30 with a processor 32. The processor 32 is connected to the supply device 26 via control lines 31. In this way, the vibration generator 11 may be actuated with different control commands from various control programs or control modes, so that different vibrations or oscillations can be generated with the vibration generator 11. The control device 30 contains a memory unit 33 in which the various control commands or control modes are stored, and from which the control commands can be retrieved to the processor 32. The control device 30 has an interface 34, with which the control device 30 of the stimulation device 10 may be connected to an external end device, for example a computer, tablet or smartphone, and control commands may be exchanged for controlling the eccentric element 19 of the vibration generator 11.

FIG. 3 shows the stimulation device 10 according to the invention described previously and a base 24. The stimulation device 10 with vibration generator 11 is mounted on the base 24. For this purpose, the spring elements 13 and 15 of the stimulation device 10 which are connected to the vibration generator 11 have a fastening section 23. The fastening section 23 comprises fastening means 29, which connect the spring elements 13 and 15 to the base 24 and attach them to the base 24. The vibration generator 11 is arranged so as to be adjustable on the base 24. In this way, the spatial position of the vibration generator 11 relative to the base can be changed. To achieve this, the vibration generator 11 is displaceable along a first longitudinal axis on the base 24. The vibration generator 11 is also mounted on the base 24 so as to be rotatable about the fastening means 29. The base 24 has a base opening 25. At least a section of the vibration generator housing 16 is inserted in the base opening 25, in such manner that the vibration generator housing 16 at least partly passes through the base opening 25. Besides the eccentric element 19, the vibration generator 11 also has a spike-shaped stimulation unit 21, which is arranged inside the vibration generator housing 16 and protrudes from the base opening 25. The spike-shaped stimulation unit 21 is arranged on the side of the base 24 opposite the fastening section 23. The vibration generator housing 16 and the ends of the first spring elements 13 and the second spring element 15 which are each attached to the vibration generator housing 16 are surrounded by an insulation layer 22. The insulation layer 22 is made from synthetic resin. The insulation layer 22 may also be designed as vibration insulation, in which case it is made from elastic material, such as a rubber material. The respective ends of the first spring element 13 and the second spring element 15 opposite the vibration generator housing 16 and in the region of the fastening section 23 are each connected to the battery unit or accumulator unit 28 via supply lines 27. Alternatively, the spring elements 13 and 15 are connected directly to the battery unit or accumulator unit 28 (not shown).

FIG. 4 shows the stimulation device 10 described here with a vibration generator 11 which has a vibration element 19a for generating vibrations or oscillations inside the vibration generator 11. The vibration element 19a is accelerated in the drive direction 18a by means of a short electromagnetic pulse and is then moved back to its starting position. For this purpose, a drive actuator 20a is arranged inside the vibration generator housing 16 of the vibration generator 11 and is connected to the control device 30 which is also located there in order to exchange control commands. The mass moment of inertia produced thereby causes a simple harmonic oscillation. The vibration created by a commercially available Vibracall element is not initiated until an oscillation frequency of about 200 Hz is reached. The drive direction 18a shown here is only one of many conceivable directions of movement of the vibration element 19a.

Accordingly, a conventional vibration generator 11 such as a Vibracall, for example, in which noise generation is typically intended or noise generation is welcome as part of the vibration process or oscillation process, is usable as a noiseless stimulator for nerve cell ends.

FIG. 5 shows the prosthesis device 50 according to the invention with the stimulation device 10 as described here for stimulating reinnervated nerve cell ends 62 on a nerve area section 61 typical of the physiognomy on the skin surface 60 of the prosthesis wearer. The stimulation device 10 comprises the vibration generator 11 with the eccentric element 19 as described here and together with the spring elements 13 and 15, which function as decoupling elements to prevent the vibrations or oscillations from the vibration generator 11 from acting on the prosthesis shaft 52. In this context, the prosthesis shaft 52 assumes the position and properties of the base as described.

The prosthesis shaft 52 has a positioning section 53 for positioning the vibration generator 11 on the reinnervated nerve cell ends 62 or on the nerve area sections 61 typical of the physiognomy of the skin surface 60. A prosthesis opening 54 is provided in the positioning section 53, and the vibration generator 11 is positioned therein. The stimulation device 10 is fastened to the prosthesis shaft 52 of the prosthesis device 50. For this purpose, the spring element 13 and 15 have a fastening section 23 with a fastening means 29. The vibration generator 11 is thus attached to the prosthesis shaft 52 by means of the spring elements 13 and 15 and the fastening means 29. The vibration generator 11 arranged adjustably on the prosthesis shaft 52. Then, the spatial position of the vibration generator 11 may be changed relative to the prosthesis shaft 52. The vibration generator 11 is displaceable along a first longitudinal axis on the prosthesis shaft 52. Additionally, the vibration generator 11 is arranged on the prosthesis shaft 52 so as to be rotatable about the fastening means 29.

The battery unit or accumulator unit 28 together with the charge connector 35 is arranged on the prosthesis shaft 52 and connected electrically to the vibration generator 11. The control device 30 as described previously for controlling the vibration generator 11 is also arranged on the prosthesis shaft 52, and the vibration generator 11 is connected electrically to the control device 30. The control device 30 has an interface 34 to allow connection of an end device, such as a computer. Alternatively, the vibration generator 11 is equipped with the vibration element described here (FIG. 4)—not shown.

FIG. 6 shows the prosthesis device 50 described previously with several of the stimulation devices 10 as described here for stimulating reinnervated nerve cell ends and/or nerve area sections 61 typical of the physiognomy on the skin surface 60 of the prosthesis wearer. The multiple stimulation devices 10 are each fastened to the prosthesis shaft 52 by their own fastening means 29, so the vibrations or oscillations of the individual vibration generators of the respective stimulation device 10 can be damped independently of each other and decoupled from the prosthesis shaft 52. The control device 30 and the battery unit or accumulator unit 28 are arranged on the prosthesis shaft 52 and connected to each other via the control line 31. The multiple stimulation devices 10 are each arranged individually on positioning section 53 according to the location of the nerve area sections 61 typical of the physiognomy of the prosthesis wearer, can be positioned with the respective fastening means 29 and are electrically connected to the battery unit or accumulator unit 28 by means of the detachable supply lines 27. A seal insert 57, made from a silicone material for example, may be interposed between the prosthesis opening 54 and the vibration generator 11.

Additionally, an electrical supply device 26 is present on the prosthesis shaft 50, the electrical supply device 26 being electrically connected to the vibration generators 11. The electrical supply device 26 is equipped with a charge coupler 36, with which the electrical supply device 26 is inductively rechargeable using a separate charger (not shown).

The stimulation device 10 described here may also be used as a signal generator in a communication apparatus, wherein this is typically a portable communication apparatus. In such a case, the stimulation device 10, as described in FIGS. 1 to 3, is arranged on the housing or the base of the mobile phone or Tablet and acts on its surroundings, for example on the user's skin surface. The housing of the communication apparatus includes a housing opening or base opening, in which the vibration generator of the stimulation device 10 is arranged. In this context, at least a section of the vibration generator is disposed inside the housing opening, so at least a section of the vibration generator extends entirely through the housing opening. In the example shown, in which the vibration generator is cylindrical, a section of the cylindrical surface of the cylinder extends through the housing opening (vertical arrangement). A soft and/or elastic seal, made from a silicone mass for example, is arranged between the vibration generator and the housing opening.

Alternatively, the vibration generator as described can also be installed in the housing opening of the communication apparatus in a position that is rotated through 90° compared with the embodiment shown in FIGS. 1 to 3. The vibration generator then extends with the a section of the mantle surface through the housing opening (horizontal arrangement). A soft and/or elastic seal, made from a silicone mass for example, is arranged between the vibration generator and the housing opening.

The communication apparatus may further be equipped with a stimulation device 10 with a horizontally arranged vibration generator and a vertically arranged vibration generator, each of which is disposed in its own housing opening in in the communication apparatus. The communication apparatus is equipped with a selection switching unit, an analogue switch or a digital switch for selecting the respective vibration generators. In this case, the user can use the selection switching unit to select either the horizontally arranged vibration generator or the vertically arranged vibration generator as the signal generator.

### REFERENCE LIST

10 Stimulation device
11 Vibration generator
12 Decoupling element
13 1st spring element
14 Further decoupling element
15 2nd spring element
16 Vibration generator housing
17 Drive shaft
18 Drive direction of 19
18a Drive direction of 19a
19 Eccentric element
19a Vibration element
20 Drive motor
20a Drive actuator
21 Stimulation unit
22 Insulation layer
23 Fastening section
24 Base
25 Base opening
26 Supply device
27 Supply line
28 Battery unit
29 Fastening means
30 Control device
31 Control line
32 Processor
33 Memory unit
34 Interface
35 Charge connector
36 Charge coupler
50 Prosthesis device
52 Prosthesis shaft
53 Positioning section
54 Prosthesis opening
57 Seal insert
60 Skin surface
61 Nerve area section typical of the physiognomy
62 Reinnervated nerve cell ends

1. A stimulation device (10) for stimulating reinnervated nerve cell ends (62), comprising:
at least one vibration generator (11),
wherein the at least one vibration generator (11) is connected to at least one decoupling element (12) in vibration-proof manner.

2. The stimulation device (10) according to claim 1, further comprising a base (24) on which the at least one vibration generator (11) is arranged, wherein the at least one vibration generator (11) is connected to the base (24) in positionally fixed manner via the at least one decoupling element (12).

3. The stimulation device (10) according to claim 1, further comprising: at least one further decoupling element (14), which is connected to the at least one vibration generator (11).

4. The stimulation device (10) according to claim 1, wherein at least the at least one decoupling element (12) is electrically conductive.

5. The stimulation device (10) according to claim 1, further comprising at least one of a supply device (26) and a control device (30), wherein at least the at least one vibration generator (11) is electrically connected to the at least one of the supply device (26) and the control device (30).

6. The stimulation device (10) according to claim 1, wherein the at least one vibration generator (11) has an eccentric element (19) or a vibration element (19a).

7. The stimulation device (10) according to claim 6, wherein the at least one vibration generator (11) has a vibration generator housing (16).

8. The stimulation device (10) according to claim 1, wherein the at least one vibration generator (11) has a stimulation unit (21) for stimulating nerve cell ends.

9. The stimulation device (10) according to claim 1, wherein at least a section of the at least one vibration generator (11) is surrounded by an insulation layer (22).

10. A prosthesis device (50), comprising:
at least one stimulation device (10) for delivering information to nerve cell ends, for stimulating the nerve cell ends, including nerve area sections (61) typical of the physiognomy, the at least one stimulation device (10) including:
at least one vibration generator (11), wherein the at least one vibration generator (11) is connected to at least one decoupling element (12, 14) in vibration-proof manner;
a prosthesis shaft (52); and
at least one vibration generator (11),
wherein the at least one vibration generator (11) is connected in positionally fixed manner to the prosthesis shaft (52) via at least one decoupling element (12, 14).

11. The prosthesis device (50) according to claim 10, further comprising:
an electrical supply device (26) provided on the prosthesis shaft (52),
wherein the electrical supply device (26) is electrically connected to the stimulation device (10), including to the at least one vibration generator (11).

12. The prosthesis device (50) according to claim 10, further comprising a control device (30) provided on the prosthesis shaft (52) for controlling the at least one vibration generator (11), wherein the at least one vibration generator (11) is electrically connected to the control device (30).

13. The prosthesis device (50) according to claim 10, wherein the prosthesis shaft (52) has at least one positioning section (53) for positioning the at least one vibration generator (11), and the positioning section (53) has at least one prosthesis opening (54).

14. A vibration generator (11), comprising:
at least one eccentric element (19) and/or at least one vibration element (19a) for stimulating nerve cell ends by a base (24) in a vibration-decoupled manner.

15. (canceled)

16. The stimulation device (10) according to claim 3, wherein at least one of the at least one decoupling element (12) and the at least one further decoupling element (14) is a spring element (13, 15).

17. The stimulation device (10) according to claim 6, wherein the at least one vibration generator (11) has a drive motor (20) for driving the eccentric element (19) in rotating manner or a drive actuator (20a) for driving the vibration element (19a) in oscillating manner.

18. The stimulation device (10) according to claim 7, wherein the eccentric element (19) or the vibration element (19a) is arranged in the vibration generator housing (16).

19. The stimulation device (10) according to claim 8, wherein the stimulation unit (21) is constructed in the form of a spike.

20. The stimulation device (10) according to claim 11, wherein at least one decoupling element (12, 14) is arranged between the electrical supply device (26) and the at least one vibration generator (11) and is electrically connected to the electrical supply device (26) and to the at least one vibration generator (11).

21. The vibration generator (11) according to claim 14, comprising
at least one eccentric element (19) and/or at least one vibration element (19a) for stimulating nerve cell ends by a prosthesis shaft (52) in a vibration-decoupled manner and/or for delivering information from reinnervated nerve cell ends (62).
